(19) United States
(12) Patent Application Publication
(10) Pub. No.: US 2022/0223289 A1
(43) Pub. Date: Jul. 14, 2022

(54) MEANS AND METHODS FOR ASSESSING MULTIPLE SCLEROSIS (MS)

(71) Applicant: Hoffmann-La Roche Inc., Little Falls, NJ (US)

(72) Inventors: Florian LIPSMEIER, Basel (CH); Cedric Andre Marie Vincent Geoffrey SIMILLION, Lutzelfluh-Goldbach (CH); Michael LINDEMANN, Schopfheim (DE); Alf SCOTLAND, Zuerich (CH)

(73) Assignee: Hoffmann-La Roche Inc., Little Falls, NJ (US)

(21) Appl. No.: 17/708,328

(22) Filed: Mar. 30, 2022

Related U.S. Application Data
(63) Continuation of application No. PCT/EP2020/077208, filed on Sep. 29, 2020.

(30) Foreign Application Priority Data
Sep. 30, 2019 (EP) 19200549.4

(51) Int. Cl. G16H 50/20; G01N 33/564; G01N 33/68

(52) U.S. Cl. CPC G16H 50/20; G01N 2800/285; G01N 33/6896; G01N 33/564

(57) ABSTRACT

The present invention relates to the field of disease tracking. Specifically, it relates to a method for predicting the total motor score (EDSS) in a subject suffering from multiple sclerosis (MS) comprising the steps of determining at least one performance parameter from a dataset of measurements of active and passive gait and posture capabilities and cognitive capabilities from said subject, comparing the determined at least one performance parameter to a reference obtained from a computer-implemented regression model generated on training data using random forest (RF) analysis, and predicting the EDSS of the subject based on said comparison. The present invention also relates to a mobile device and/or a remote device as well as software which is tangibly embedded to one of the devices and carries out the method of the invention, wherein said mobile device and said remote device can be operatively linked to each other.

### CROSS REFERENCE TO RELATED APPLICATIONS

This application is a continuation of International Application No. PCT/EP2020/077208, filed Sep. 29, 2020, which claims priority to EP Application No. 19200549.4, filed Sep. 30, 2019, which are incorporated herein by reference in their entireties.

The present invention relates to the field of disease tracking and potentially even diagnostics. Specifically, it relates to a method for predicting the total motor score (EDSS) in a subject suffering from multiple sclerosis (MS) comprising the steps of determining at least one performance parameter from a dataset of measurements of active and passive gait and posture capabilities and cognitive capabilities from said subject, comparing the determined at least one performance parameter to a reference obtained from a computer-implemented regression model generated on training data, in an embodiment using random forest (RF) analysis, with the at least one performance parameters, and predicting the EDSS of the subject based on said comparison. The present invention also relates to a mobile device comprising a processor, at least one sensor and a database as well as software which is tangibly embedded to said device and, when running on said device, carries out the method of the invention as well as a system comprising a mobile device comprising at least one sensor and a remote device comprising a processor and a database as well as software which is tangibly embedded to said device and, when running on said device, carries out the method of the invention, wherein said mobile device and said remote device are operatively linked to each other. Furthermore, the invention contemplates the use of the aforementioned mobile device or system for predicting the EDSS in a subject suffering from MS using at least one performance parameter from a dataset of measurements of active and passive gait and posture capabilities and cognitive capabilities from said subject.

Multiple sclerosis (MS) is a severe neurodegenerative disease which at present cannot be cured. Affected by this disease are approximately 2 to 3 million individuals worldwide. It is the most common disease of the central nervous system (CNS) that causes prolonged and severe disability in young adults. There is evidence supporting the concept that a B- and T cell-mediated inflammatory process against self-molecules within the white matter of the brain and spinal cord causes the disease. However, its etiology is still not well understood. It has been found that myelin-reactive T cells are present in both MS patients and healthy individuals.

Accordingly, the primary abnormality in MS may involve more likely an impaired regulatory mechanisms leading to an enhanced T cell activation status and less stringent activation requirements. The pathogenesis of MS includes activation of encephalitogenic, i.e. autoimmune myelin-specific T cells outside the CNS, followed by an opening of the blood-brain barrier, T cell and macrophage infiltration, microglia activation and demyelination. The latter causes irreversible neuronal damage (see, e.g., Aktas 2005, Neuron 46, 421-432, Zamvil 2003, Neuron 38: 685-688).

It was shown more recently that besides T cells, B lymphocytes (expressing CD20 molecule) may play a central role in MS and influence the underlying pathophysiology through at least four specific functions:
1. Antigen presentation: B cells can present self neuroantigens to T cells and activate them.
2. Cytokine production: B cells in patients with MS produce abnormal proinflammatory cytokines, which can activate T cells and other immune cells.
3. Autoantibody production: B cells produce autoantibodies that may cause tissue damage and activate macrophages and natural killer (NK) cells.
4. Follicle-like aggregate formation: B cells are present in ectopic lymphoid follicle-like aggregates, linked to microglia activation, local inflammation, and neuronal loss in the nearby cortex.

Although there is sound knowledge about the mechanisms responsible for the encephalitogenicity, far less is known regarding the control mechanisms for regulating harmful lymphocyte responses into and within the CNS in a subject.

MS diagnosis is based at present on clinical investigations by a medical practitioner. Such investigations involve testing of the capabilities of a patient for certain physical activities. Several tests have been developed and are routinely applied by medical practitioners. These tests aim at assessing walking, balance, and other motoric abilities. Examples of currently applied tests are the Expanded Disability Status Scale (EDSS) or Multiple Sclerosis Functional Composite (MSFC). These tests require the presence of a medical practitioner for evaluation and assessment purposes and are currently performed ambulant at doctor's offices or hospitals. Very recently, there have been some efforts in monitoring MS patients using smartphone devices in order to collect data of MS patients in a natural setting (Bove 2015, Neurol Neuroimmunol Neuroinflamm 2 (6):e162).

Further, diagnostic tools are used in MS diagnosis. Such tools include neuroimaging, analysis of cerebrospinal fluid and evoked potentials. Magnetic resonance imaging (MRI) of the brain and spinal cord can visualize demyelination (lesions or plaques). Contrast agents containing gadolinium can be administered intravenously to mark active plaques and, differentiate acute inflammation from the existence of older lesions which are not associated with symptoms at the moment of the evaluation. The analysis of cerebrospinal fluid obtained from a lumbar puncture can provide evidence of chronic inflammation of the central nervous system. The cerebrospinal fluid can be analyzed for oligoclonal immunoglobulin bands, which are an inflammation marker present in 75-85% of people with MS (Link 2006, J Neuroimmunol. 180 (1-2): 17-28). However, none of the aforementioned techniques is specific to MS. Therefore, ascertainment of diagnosis may require repetition of clinical and MRI investigations to demonstrate dissemination in space and in time of the disease which is a prerequisite to MS diagnosis.

There are several treatments approved by regulatory agencies for relapsing-remitting multiple sclerosis which shall modify the course of the disease. These treatments include interferon beta-1a, interferon beta-1b, glatiramer acetate, mitoxantrone, natalizumab, fingolimod, teriflunomide, dimethyl fumarate, alemtuzumab, and daclizumab. The interferons and glatiramer acetate are first-line treatments that reduce relapses by approximately 30% (see, e.g., Tsang 2011, Australian family physician 40 (12): 948-55). Natalizumab reduces the relapse rate more than the interferons, however, due to issues of adverse effects it is a second-line agent reserved for those who do not respond to other treatments or patients with severe disease (see, e.g., Tsang 2011, loc. cit.). Treatment of clinically isolated syndrome (CIS) with interferons decreases the chance of progressing to clinically definite MS (Compston 2008, Lancet 372(9648): 1502-17). Efficacy of interferons and glatiramer acetate in children has been estimated to be roughly equivalent to that of adults (Johnston 2012, Drugs 72 (9): 1195-211).

Recently, new monoclonal antibodies such as ocrelizumab, alemtuzumab and daclizumab have shown potential as therapeutics for MS. The anti-CD20 B-cell targeting monoclonal antibody ocrelizumab has shown beneficial effects in both relapsing and primary progressive forms of MS in one phase 2 and 3 phase III trials (NCT00676715, NCT01247324, NCT01412333, NCT01194570)

MS is a clinically heterogeneous inflammatory disease of the CNS. Therefore, diagnostic tools are needed that allow a reliable diagnosis and identification of the present disease status and can, thus, aid an accurate treatment, in particular, for those patients suffering for progressing forms of MS.

For MS management, the disability status needs to be determined. The EDSS is a scoring system for classifying patients according to their disability status and, thus, allows for determining the need of assistance and/or support.

The EDSS is a score based on quantitative assessment of the disabilities in subjects suffering from MS (Krutzke 1983). The EDSS is based on a neurological examination by a clinician, although versions of the scoring system for self-administration also exist (Collins 2016). The EDSS quantifies disability in eight functional systems by assigning a Functional System Score (FSS) in each of these functional systems.

The technical problem underlying the present invention may be seen in the provision of means and methods complying with the aforementioned needs. The technical problem is solved by the embodiments characterized in the claims and described herein below.

Thus, the invention relates to a method for predicting the expanded disability status scale (EDSS) in a subject suffering from multiple sclerosis (MS) comprising the steps of:
a) determining at least one performance parameter from a dataset of measurements of active and passive gait and posture capabilities and cognitive capabilities from said subject;
b) comparing the determined at least one performance parameter to a reference obtained from a computer-implemented regression model generated on training data, in an embodiment using random forest (RF) analysis, with the at least one performance parameters; and
c) predicting the EDSS of the subject based on said comparison.

The method is, typically, a computer implemented method, i.e. the steps a) to c) are carried out in an automated manner by use of a data processing device. Details are also found herein below and in the accompanying Examples.

In some embodiments, the method may also comprise prior to step (a) the step of obtaining from the subject using a mobile device a dataset of measurements of active and passive gait and posture capabilities and cognitive capabilities from said subject during predetermined activity performed by the subject or during a predetermined time window. However, typically the method is an ex vivo method carried out on an existing dataset of measurements from a subject which does not require any physical interaction with the said subject.

The method as referred to in accordance with the present invention includes a method which essentially consists of the aforementioned steps or a method which may include additional steps.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “typically”, and “more typically” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

The method may be carried out on the mobile device by the subject once the dataset of pressure measurements has been acquired. Thus, the mobile device and the device acquiring the dataset may be physically identical, i.e. the same device. Such a mobile device shall have a data acquisition unit which typically comprises means for data acquisition, i.e. means which detect or measure either quantitatively or qualitatively physical and/or chemical parameters and transform them into electronic signals transmitted to the evaluation unit in the mobile device used for carrying out the method according to the invention. The data acquisition unit comprises means for data acquisition, i.e. means which detect or measure either quantitatively or qualitatively physical and/or chemical parameters and transform them into electronic signals transmitted to the device being remote from the mobile device and used for carrying out the method according to the invention. Typically, said means for data acquisition comprise at least one sensor. It will be understood that more than one sensor can be used in the mobile device, i.e. at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. The evaluation unit typically comprises a processor and a database as well as software which is tangibly embedded to said device and, when running on said device, carries out the method of the invention. More typically, such a mobile device may also comprise a user interface, such as a screen, which allows for providing the result of the analysis carried out by the evaluation unit to a user.

Alternatively, it may be carried out on a device being remote with respect to the mobile device that has been used to acquire the said dataset. In this case, the mobile device shall merely comprise means for data acquisition, i.e. means which detect or measure either quantitatively or qualitatively physical and/or chemical parameters and transform them into electronic signals transmitted to the device being remote from the mobile device and used for carrying out the method according to the invention. Typically, said means for data acquisition comprise at least one sensor. It will be understood that more than one sensor can be used in the mobile device, i.e. at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors, GPS, Balistocardiography, and the like. Thus, the mobile device and the device used for carrying out the method of the invention may be physically different devices. In this case, the mobile device may correspond with the device used for carrying out the method of the present invention by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of the present invention, the only requirement is the presence of a dataset of measurements obtained from a subject using a mobile device. The said dataset may also be transmitted or stored from the acquiring mobile device on a permanent or temporary memory device which subsequently can be used to transfer the data to the device used for carrying out the method of the present invention. The remote device which carries out the method of the invention in this setup typically comprises a processor and a database as well as software which is tangibly embedded to said device and, when running on said device, carries out the method of the invention. More typically, the said device may also comprise a user interface, such as a screen, which allows for providing the result of the analysis carried out by the evaluation unit to a user.

The term “predicting” as used herein refers to determining the EDSS based on at least one performance parameter determined from measured datasets and a preexisting correlation of such performance parameter(s) and the EDSS rather than by determining the EDSS directly. As will be understood by those skilled in the art, such a prediction, although preferred to be, may usually not be correct for 100% of the investigated subjects. The term, however, requires that the EDSS can be correctly predicted in a statistically significant portion of subjects. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. The term also encompasses any kind of diagnosing, monitoring or staging of MS based on EDSS and, in particular, relates to assessing, diagnosing, monitoring and/or staging of any symptom or progression of any symptom associated with MS.

The term “multiple sclerosis (MS)” as used herein relates to disease of the central nervous system (CNS) that typically causes prolonged and severe disability in a subject suffering therefrom. There are four standardized subtype definitions of MS which are also encompassed by the term as used in accordance with the present invention: relapsing-remitting, secondary progressive, primary progressive and progressive relapsing. The term relapsing forms of MS is also used and encompasses relapsing-remitting and secondary progressive MS with superimposed relapses. The relapsing-remitting subtype is characterized by unpredictable relapses followed by periods of months to years of remission with no new signs of clinical disease activity. Deficits suffered during attacks (active status) may either resolve or leave sequelae. This describes the initial course of 85 to 90% of subjects suffering from MS. Secondary progressive MS describes those with initial relapsing-remitting MS, who then begin to have progressive neurological decline between acute attacks without any definite periods of remission. Occasional relapses and minor remissions may appear. The median time between disease onset and conversion from relapsing remitting to secondary progressive MS is about 19 years. The primary progressive subtype describes about 10 to 15% of subjects who never have remission after their initial MS symptoms. It is characterized by progressive of disability from onset, with no, or only occasional and minor, remissions and improvements. The age of onset for the primary progressive subtype is later than other subtypes. Progressive relapsing MS describes those subjects who, from onset, have a steady neurological decline but also suffer clear superimposed attacks. It is now accepted that this latter progressive relapsing phenotype is a variant of primary progressive MS (PPMS) and diagnosis of PPMS according to McDonald 2010 criteria includes the progressive relapsing variant.

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder, sexual and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard text books of medicine and neurology.

The term “progressing MS” as used herein refers to a condition, where the disease and/or one or more of its symptoms get worse over time. Typically, the progression is accompanied by the appearance of active statuses. The said progression may occur in all subtypes of the disease. However, typically “progressing MS” shall be determined in accordance with the present invention in subjects suffering from relapsing-remitting MS.

For MS management, the disability status needs to be determined. The expanded disability status scale (EDSS) is a scoring system for classifying patients according to their disability status and, thus, allows for determining the need of assistance and/or support.

The term “expanded disability status scale (EDSS)” as used herein, thus, refers to a score based on quantitative assessment of the disabilities in subjects suffering from MS (Krutzke 1983). The EDSS is based on a neurological examination by a clinician. The EDSS quantifies disability in eight functional systems by assigning a Functional System Score (FSS) in each of these functional systems. The functional systems are the pyramidal system, the cerebellar system, the brainstem system, the sensory system, the bowel and bladder system, the visual system, the cerebral system and other (remaining) systems. EDSS steps 1.0 to 4.5 refer to subjects suffering from MS who are fully ambulatory, EDSS steps 5.0 to 9.5 characterize those with impairment to ambulation.

The clinical meaning of each possible result is the following:
0.0: Normal Neurological Exam
1.0: No disability, minimal signs in 1 FS
1.5: No disability, minimal signs in more than 1 FS
2.0: Minimal disability in 1 FS
2.5: Mild disability in 1 or Minimal disability in 2 FS
3.0: Moderate disability in 1 FS or mild disability in 3-4 FS, though fully ambulatory
3.5: Fully ambulatory but with moderate disability in 1 FS and mild disability in 1 or 2 FS; or moderate disability in 2 FS; or mild disability in 5 FS
4.0: Fully ambulatory without aid, up and about 12 hrs a day despite relatively severe disability. Able to walk without aid 500 meters
4.5: Fully ambulatory without aid, up and about much of day, able to work a full day, may otherwise have some limitations of full activity or require minimal assistance. Relatively severe disability. Able to walk without aid 300 meters
5.0: Ambulatory without aid for about 200 meters. Disability impairs full daily activities
5.5: Ambulatory for 100 meters, disability precludes full daily activities
6.0: Intermittent or unilateral constant assistance (cane, crutch or brace) required to walk 100 meters with or without resting
6.5: Constant bilateral support (cane, crutch or braces) required to walk 20 meters without resting
7.0: Unable to walk beyond 5 meters even with aid, essentially restricted to wheelchair, wheels self, transfers alone; active in wheelchair about 12 hours a day
7.5: Unable to take more than a few steps, restricted to wheelchair, may need aid to transfer; wheels self, but may require motorized chair for full day's activities
8.0: Essentially restricted to bed, chair, or wheelchair, but may be out of bed much of day; retains self-care functions, generally effective use of arms
8.5: Essentially restricted to bed much of day, some effective use of arms, retains some self-care functions
9.0: Helpless bed patient, can communicate and eat
9.5: Unable to communicate effectively or eat/swallow
10.0: Death due to MS

The term “subject” as used herein relates to animals and, typically, to mammals. In particular, the subject is a primate and, most typically, a human. The subject in accordance with the present invention shall suffer from or shall be suspected to suffer from MS, i.e. it may already show some or all of the symptoms associated with the said disease.

The term “at least one” means that one or more performance parameters may be determined in accordance with the invention, i.e. at least two, at least three, at least four or even more different performance parameters. Thus, there is no upper limit for the number of different performance parameters which can be determined in accordance with the method of the present invention.

Typically, however, there will be 32 different performance parameters used. More typically, the parameter(s) are selected from datasets of measurements of active and passive gait and posture capabilities and cognitive capabilities. Typically, said measurements of active and passive gait and posture capabilities and cognitive capabilities comprise measurements relating to movement characteristics, in particular, movement pattern or time required for performing a movement task, or accuracy, time or correctness of performing a cognitive task.

The term “performance parameter” as used herein refers to a parameter which is indicative for the capability of a subject to carry out a certain activity. Typically, the performance parameter is a movement parameter, in particular, a parameter indicative for a movement pattern or time required for performing a movement task, or a parameter indicative for accuracy, time or correctness of performing a cognitive task. More typically, the performance parameter is selected from performance parameters indicative for active and passive gait and posture capabilities and cognitive capabilities. Particular performance parameters to be used in accordance with the present invention are listed elsewhere herein in more detail (see Table 1, below). In an embodiment, the expression “gait” is used herein for “active and passive gait”; similarly, in an embodiment, the term “posture” may be used for “active and passive posture”.

The term “dataset of measurements” refers to the entirety of data which has been acquired by the mobile device from a subject during measurements or any subset of said data useful for deriving the performance parameter.

The at least one performance parameter can be typically determined from datasets of measurements collected from the subject during carrying out the following activities. The following tests are typically computer-implemented on a data acquisition device such as a mobile device as specified elsewhere herein.

(1) Tests for Passive Monitoring of Gait and Posture

The mobile device is, typically, adapted for performing or acquiring data from passive monitoring of all or a subset of activities. In particular, the passive monitoring shall encompass monitoring one or more activities performed during a predefined window, such as one or more days or one or more weeks, selected from the group consisting of: measurements of gait, the amount of movement in daily routines in general, the types of movement in daily routines, general mobility in daily living and changes in moving behavior.

Typical passive monitoring performance parameters of interest:
a. frequency and/or velocity of walking;
b. amount, ability and/or velocity to stand up/sit down, stand still and balance
c. number of visited locations as an indicator of general mobility;
d. types of locations visited as an indicator of moving behavior.

(2) Test for Cognitive Capabilities: The eSDMT Test

The mobile device is also, typically, adapted for performing or acquiring a data from an computer-implemented Symbol Digit Modalities Test (eSDMT). The conventional paper SDMT version of the test consists of a sequence of 120 symbols to be displayed in a maximum 90 seconds and a reference key legend (3 versions are available) with 9 symbols in a given order and their respective matching digits from 1 to 9. The smartphone-based eSDMT is meant to be self-administered by patients and will use a sequence of symbols, typically, the same sequence of 110 symbols, and a random alternation (from one test to the next) between reference key legends, typically, the 3 reference key legends, of the paper/oral version of SDMT. The eSDMT similarly to the paper/oral version measures the speed (number of correct paired responses) to pair abstract symbols with specific digits in a predetermined time window, such as 90 seconds time. The test is, typically, performed weekly but could alternatively be performed at higher (e.g. daily) or lower (e.g. bi-weekly) frequency. The test could also alternatively encompass more than 110 symbols and more and/or evolutionary versions of reference key legends. The symbol sequence could also be administered randomly or according to any other modified pre-specified sequence.

Typical eSDMT performance parameters of interest:
1. Number of correct responses
   a. Total number of overall correct responses (CR) in 90 seconds (similar to oral/paper SDMT)
   b. Number of correct responses from time 0 to 30 seconds (CR₀₋₃₀)
   c. Number of correct responses from time 30 to 60 seconds (CR₃₀₋₆₀)
   d. Number of correct responses from time 60 to 90 seconds (CR₆₀₋₉₀)
   e. Number of correct responses from time 0 to 45 seconds (CR₀₋₄₅)
   f. Number of correct responses from time 45 to 90 seconds (CR₄₅₋₉₀)
   g. Number of correct responses from time i to j seconds (CRᵢ₋ⱼ), where i,j are between 1 and 90 seconds and i<j.
2. Number of errors
   a. Total number of errors (E) in 90 seconds
   b. Number of errors from time 0 to 30 seconds (E₀₋₃₀)
   c. Number of errors from time 30 to 60 seconds (E₃₀₋₆₀)
   d. Number of errors from time 60 to 90 seconds (E₆₀₋₉₀)
   e. Number of errors from time 0 to 45 seconds (E₀₋₄₅)
   f. Number of errors from time 45 to 90 seconds (E₄₅₋₉₀)
   g. Number of errors from time i to j seconds (Eᵢ₋ⱼ), where i,j are between 1 and 90 seconds and i<j.
3. Number of responses
   a. Total number of overall responses (R) in 90 seconds
   b. Number of responses from time 0 to 30 seconds (R₀₋₃₀)
   c. Number of responses from time 30 to 60 seconds (R₃₀₋₆₀)
   d. Number of responses from time 60 to 90 seconds (R₆₀₋₉₀)
   e. Number of responses from time 0 to 45 seconds (R₀₋₄₅)
   f. Number of responses from time 45 to 90 seconds (R₄₅₋₉₀)
4. Accuracy rate
   a. Mean accuracy rate (AR) over 90 seconds: AR=CR/R
   b. Mean accuracy rate (AR) from time 0 to 30 seconds: AR₀₋₃₀=CR₀₋₃₀/R₀₋₃₀
   c. Mean accuracy rate (AR) from time 30 to 60 seconds: AR₃₀₋₆₀=CR₃₀₋₆₀/R₃₀₋₆₀
   d. Mean accuracy rate (AR) from time 60 to 90 seconds: AR₆₀₋₉₀=CR₆₀₋₉₀/R₆₀₋₉₀
   e. Mean accuracy rate (AR) from time 0 to 45 seconds: AR₀₋₄₅=CR₀₋₄₅/R₀₋₄₅
   f. Mean accuracy rate (AR) from time 45 to 90 seconds: AR₄₅₋₉₀=CR₄₅₋₉₀/R₄₅₋₉₀
5. End of task fatigability indices
   a. Speed Fatigability Index (SFI) in last 30 seconds: SFI₆₀₋₉₀=CR₆₀₋₉₀/max (CR₀₋₃₀, CR₃₀₋₆₀)
   b. SFI in last 45 seconds: SFI₄₅₋₉₀=CR₄₅₋₉₀/CR₀₋₄₅
   c. Accuracy Fatigability Index (AFI) in last 30 seconds: AFI₆₀₋₉₀=AR₆₀₋₉₀/max (AR₀₋₃₀, AR₃₀₋₆₀)
   d. AFI in last 45 seconds: AFI₄₅₋₉₀=AR₄₅₋₉₀/AR₀₋₄₅
6. Longest sequence of consecutive correct responses
   a. Number of correct responses within the longest sequence of overall consecutive correct responses (CCR) in 90 seconds
   b. Number of correct responses within the longest sequence of consecutive correct responses from time 0 to 30 seconds (CCR₀₋₃₀)
   c. Number of correct responses within the longest sequence of consecutive correct responses from time 30 to 60 seconds (CCR₃₀₋₆₀)
   d. Number of correct responses within the longest sequence of consecutive correct responses from time 60 to 90 seconds (CCR₆₀₋₉₀)
   e. Number of correct responses within the longest sequence of consecutive correct responses from time 0 to 45 seconds (CCR₀₋₄₅)
   f. Number of correct responses within the longest sequence of consecutive correct responses from time 45 to 90 seconds (CCR₄₅₋₉₀)
7. Time gap between responses
   a. Continuous variable analysis of gap (G) time between two successive responses
   b. Maximal gap (GM) time elapsed between two successive responses over 90 seconds
   c. Maximal gap time elapsed between two successive responses from time 0 to 30 seconds (GM₀₋₃₀)
   d. Maximal gap time elapsed between two successive responses from time 30 to 60 seconds (GM₃₀₋₆₀)
   e. Maximal gap time elapsed between two successive responses from time 60 to 90 seconds (GM₆₀₋₉₀)
   f. Maximal gap time elapsed between two successive responses from time 0 to 45 seconds (GM₀₋₄₅)
   g. Maximal gap time elapsed between two successive responses from time 45 to 90 seconds (GM₄₅₋₉₀)
8. Time Gap between correct responses
   a. Continuous variable analysis of gap (Gc) time between two successive correct responses
   b. Maximal gap time elapsed between two successive correct responses (GcM) over 90 seconds
   c. Maximal gap time elapsed between two successive correct responses from time 0 to 30 seconds (GcM₀₋₃₀)
   d. Maximal gap time elapsed between two successive correct responses from time 30 to 60 seconds (GcM₃₀₋₆₀)
   e. Maximal gap time elapsed between two successive correct responses from time 60 to 90 seconds (GcM₆₀₋₉₀)
   f. Maximal gap time elapsed between two successive correct responses from time 0 to 45 seconds (GcM₀₋₄₅)
   g. Maximal gap time elapsed between two successive correct responses from time 45 to 90 seconds (GcM₄₅₋₉₀)
9. Fine finger motor skill function parameters captured during eSDMT
   a. Continuous variable analysis of duration of touchscreen contacts (Tts), deviation between touchscreen contacts (Dts) and center of closest target digit key, and mistyped touchscreen contacts (Mts) (i.econtacts not triggering key hit or triggering key hit but associated with secondary sliding on screen), while typing responses over 90 seconds
   b. Respective variables by epochs from time 0 to 30 seconds: Tts₀₋₃₀, Dts₀₋₃₀, MtS₀₋₃₀
   c. Respective variables by epochs from time 30 to 60 seconds: Tts₃₀₋₆₀, Dts₃₀₋₆₀, MtS₃₀₋₆₀
   d. Respective variables by epochs from time 60 to 90 seconds: Tts₆₀₋₉₀, Dts₆₀₋₉₀, MtS₆₀₋₉₀
   e. Respective variables by epochs from time 0 to 45 seconds: Tts₀₋₄₅, Dts₀₋₄₅, MtS₀₋₄₅
   f. Respective variables by epochs from time 45 to 90 seconds: Tts₄₅₋₉₀, Dts₄₅₋₉₀, MtS₄₅₋₉₀
10. Symbol-specific analysis of performances by single symbol or cluster of symbols
   a. CR for each of the 9 symbols individually and all their possible clustered combinations
   b. AR for each of the 9 symbols individually and all their possible clustered combinations
   c. Gap time (G) from prior response to recorded responses for each of the 9 symbols individually and all their possible clustered combinations
   d. Pattern analysis to recognize preferential incorrect responses by exploring the type of mistaken substitutions for the 9 symbols individually and the 9 digit responses individually.
   e.
11. Learning and cognitive reserve analysis
   a. Change from baseline (baseline defined as the mean performance from the first 2 administrations of the test) in CR (overall and symbol-specific as described in #9) between successive administrations of eSDMT
   b. Change from baseline (baseline defined as the mean performance from the first 2 administrations of the test) in AR (overall and symbol-specific as described in #9) between successive administrations of eSDMT
   c. Change from baseline (baseline defined as the mean performance from the first 2 administrations of the test) in mean G and GM (overall and symbol-specific as described in #9) between successive administrations of eSDMT
   d. Change from baseline (baseline defined as the mean performance from the first 2 administrations of the test) in mean Gc and GcM (overall and symbol-specific as described in #9) between successive administrations of eSDMT
   e. Change from baseline (baseline defined as the mean performance from the first 2 administrations of the test) in SFI₆₀₋₉₀ and SFI₄₅₋₉₀ between successive administrations of eSDMT
   f. Change from baseline (baseline defined as the mean performance from the first 2 administrations of the test) in AFI₆₀₋₉₀ and AFI₄₅₋₉₀ between successive administrations of eSDMT
   g. Change from baseline (baseline defined as the mean performance from the first 2 administrations of the test) in Tts between successive administrations of eSDMT
   h. Change from baseline (baseline defined as the mean performance from the first 2 administrations of the test) in Dts between successive administrations of eSDMT
   i. Change from baseline (baseline defined as the mean performance from the first 2 administrations of the test) in Mts between successive administrations of eSDMT.

(3) Tests for Active Gait and Posture Capabilities: The 5UTT and 2MWT Test

A sensor-based (e.g. accelerometer, gyroscope, magnetometer, global positioning system [GPS]) and computer implemented test for measures of ambulation performances and gait and stride dynamics, in particular, the 2-Minute Walking Test (2MWT) and the Five U-Turn Test (5UTT).

In one embodiment, the mobile device is adapted to perform or acquire data from the Two-Minute Walking Test (2MWT). The aim of this test is to assess difficulties, fatigability or unusual patterns in long-distance walking by capturing gait features in a two-minute walk test (2MWT). Data will be captured from the mobile device. A decrease of stride and step length, increase in stride duration, increase in step duration and asymmetry and less periodic strides and steps may be observed in case of disability progression or emerging relapse. Arm swing dynamic while walking will also be assessed via the mobile device. The subject will be instructed to “walk as fast and as long as you can for 2 minutes but walk safely”. The 2MWT is a simple test that is required to be performed indoor or outdoor, on an even ground in a place where patients have identified they could walk straight for as far as ≥200 meters without U-turns. Subjects are allowed to wear regular footwear and an assistive device and/or orthotic as needed. The test is typically performed daily.

Typical 2MWT performance parameters of particular interest:
1. Surrogate of walking speed and spasticity:
   a. Total number of steps detected in, e.g., 2 minutes (ΣS)
   b. Total number of rest stops if any detected in 2 minutes (ΣRs)
   c. Continuous variable analysis of walking step time (WsT) duration throughout the 2MWT
   d. Continuous variable analysis of walking step velocity (WsV) throughout the 2MWT (step/second)
   e. Step asymmetry rate throughout the 2MWT (mean difference of step duration between one step to the next divided by mean step duration): SAR=meanΔ(WsTₓ−WsTₓ₊₁)/(120/ΣS)
   f. Total number of steps detected for each epoch of 20 seconds (ΣS_{t, t+20})
   g. Mean walking step time duration in each epoch of 20 seconds: WsT_{t, t+20}=20/ΣS_{t, t+20}
   h. Mean walking step velocity in each epoch of 20 seconds: WsV_{t, t+20}=ΣS_{t, t+20}/20
   i. Step asymmetry rate in each epoch of 20 seconds: SAR_{t, t+20}=meanΔ_{t, t+20}(WsTₓ−WsTₓ₊₁)/(20/ΣS_{t, t+20})
   j. Step length and total distance walked through biomechanical modelling
2. Walking fatigability indices:
   a. Deceleration index: DI=WsV₁₀₀₋₁₂₀/max (WsV₀₋₂₀, WsV₂₀₋₄₀, WsV₄₀₋₆₀)
   b. Asymmetry index: AI=SAR₁₀₀₋₁₂₀/min (SAR₀₋₂₀, SAR₂₀₋₄₀, SAR₄₀₋₆₀)

In another embodiment, the mobile device is adapted to perform or acquire data from the Five U-Turn Test (5UTT). The aim of this test is to assess difficulties or unusual patterns in performing U-turns while walking on a short distance at comfortable pace. The 5UTT is required to be performed indoor or outdoor, on an even ground where patients are instructed to “walk safely and perform five successive U-turns going back and forward between two points a few meters apart”. Gait feature data (change in step counts, step duration and asymmetry during U-turns, U-turn duration, turning speed and change in arm swing during U-turns) during this task will be captured by the mobile device. Subjects are allowed to wear regular footwear and an assistive device and/or orthotic as needed. The test is typically performed daily.

Typical 5UTT performance parameters of interest:
1. Mean number of steps needed from start to end of complete U-turn (ΣSu)
2. Mean time needed from start to end of complete U-turn (Tu)
3. Mean walking step duration: Tsu=Tu/ΣSu
4. Turn direction (left/right)
5. Turning speed (degrees/sec)

In an embodiment, at least one performance parameter selected from the performance parameters listed in Table 1 is determined. In a further embodiment, at least two, at least three, at least four, at least five, at least ten, at least 15, at least 20, at least 25, or at least 30, performance parameters of Table 1 are determined. In a further embodiment, at least three, in a further embodiment at least five, in a further embodiment at least ten, in a further embodiment at least 15, in a further embodiment at least 20, in a further embodiment at least 25, in a further embodiment at least 30, performance parameters of Table 1 are determined. In a further embodiment all performance parameters listed Table 1 are determined.

TABLE 1
Typical performance parameters for active and passive gait
and posture capabilities and cognitive capabilities

| Performance parameter | test | Description of feature | rank |
|---|---|---|---|
| logistic step_power_mean | Passive | Average per-step power coefficient | 1 |
| (40-60 s) | Monitoring | (integral of variance in accelerometer | |
| | | radius over per-step time span) for | |
| | | gait bouts spanning 40-60 s | |
| sigmoid turns_utt | U-TURN | Number of turns | 2 |
| log10 Gc_0_15 | SDMT | Mean Timegap between correct | 3 |
| | | responses from time 0 to 15 seconds | |
| sigmoid | U-TURN | maximum turn speed | 4 |
| turn_speed_max_utt | | | |
| logistic step_power_mean | 2MWT | Average per-step power coefficient | 5 |
| | | (integral of variance in accelerometer | |
| | | radius over per-step time span) | |
| sigmoid turn_speed_min_utt | U-TURN | minimum turn speed | 6 |
| sigmoid | Passive | Variance of per-step power coefficient | 7 |
| step_power_variance | Monitoring | for gait bouts spanning 60-90 s | |
| (60-90 s) | | | |
| logistic | Passive | Variance of per-step power coefficient | 8 |
| step_power_variance | Monitoring | for gait bouts spanning 40-60 s | |
| (40-60 s) | | | |
| sigmoid step_power_mean | Passive | Average per-step power coefficient | 9 |
| (<20 s) | Monitoring | (integral of variance in accelerometer | |
| | | radius over per-step time span) for | |
| | | gait bouts spanning <20 s | |
| span_duration_s_median_utt | U-TURN | median gait bout length | 10 |
| logistic | Passive | Variance of per-step power coefficient | 11 |
| step_power_variance | Monitoring | for gait bouts spanning 20-40 s | |
| (20-40 s) | | | |
| sigmoid | Passive | Variance of per-step power coefficient | 12 |
| step_power_variance | Monitoring | for gait bouts spanning 90-120 s | |
| (90-120 s) | | | |
| sigmoid | U-TURN | median turn speed | 13 |
| turn_speed_median_utt | | | |
| logistic step_power_mean | Passive | Average per-step power coefficient | 14 |
| (60-90 s) | Monitoring | (integral of variance in accelerometer | |
| | | radius over per-step time span) for | |
| | | gait bouts spanning 60-90 s | |
| sigmoid GcM_0_15 | SDMT | Maximal Timegap between correct | 15 |
| | | responses from time 0 to 15 seconds | |
| logistic step_power_mean | Passive | Average per-step power coefficient | 16 |
| (20-40 s) | Monitoring | (integral of variance in accelerometer | |
| | | radius over per-step time span) for | |
| | | gait bouts spanning 20-40 s | |
| logistic step_power_mean | Passive | Average per-step power coefficient | 17 |
| (90-120 s) | Monitoring | (integral of variance in accelerometer | |
| | | radius over per-step time span) for | |
| | | gait bouts spanning 90-120 s | |
| CCR_0_45 | SDMT | from time 0 to 45 seconds: Number of | 18 |
| | | correct responses within the longest | |
| | | sequence of overall consecutive | |
| | | correct responses | |
| span_duration_s_max_utt | U-TURN | maximum gait bout length | 19 |
| log10 R_Symbol_9 | SDMT | Number of total responses for symbol | 20 |
| | | 9: “.-” | |
| Gc_0_30 | SDMT | Mean Timegap between correct | 21 |
| | | responses from time 0 to 30 seconds | |
| sigmoid CCR_0_15 | SDMT | from time 0 to 15 seconds: Number of | 22 |
| | | correct responses within the longest | |
| | | sequence of overall consecutive | |
| | | correct responses | |
| sigmoid GM_0_15 | SDMT | Maximal Timegap between responses | 23 |
| | | from time 0 to 15 seconds | |
| sigmoid R_0_15 | SDMT | Number of total responses from time 0 | 24 |
| | | to 15 seconds | |
| log10 CR_Symbol_8 | SDMT | Number of correct responses for | 25 |
| | | symbol 8: “)” | |
| log10 CCR_0_30 | SDMT | from time 0 to 30 seconds: Number of | 26 |
| | | correct responses within the longest | |
| | | sequence of overall consecutive | |
| | | correct responses | |
| log10 G_0_15 | SDMT | Mean Timegap between responses | 27 |
| | | from time 0 to 15 seconds | |
| sigmoid CR_0_15 | SDMT | Number of correct responses from | 28 |
| | | time 0 to 15 seconds | |
| log10 Gc_0_45 | SDMT | Mean Timegap between correct | 29 |
| | | responses from time 0 to 45 seconds | |
| log10 R_Symbol_8 | SDMT | Number of total responses for symbol | 30 |
| | | 8: “)” | |
| log10 R_0_30 | SDMT | Number of total responses from time 0 | 31 |
| | | to 30 seconds | |
| sigmoid CR_0_30 | SDMT | Number of correct responses from | 32 |
| | | time 0 to 30 seconds | |

However, in accordance with the method of the present invention, further clinical, biochemical or genetic parameters may be considered.

The term “mobile device” as used herein refers to any portable device which comprises at least a sensor and data-recording equipment suitable for obtaining the dataset of the above measurements. This may also require a data processor and storage unit as well as a display for electronically simulating a pressure measurement test on the mobile device. The data processor may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. Moreover, from the activity of the subject data shall be recorded and compiled to a dataset which is to be evaluated by the method of the present invention either on the mobile device itself or on a second device. Depending on the specific setup envisaged, it may be necessary that the mobile device comprises data transmission equipment in order to transfer the acquired dataset from the mobile device to further device. Particular well suited as mobile devices according to the present invention are smartphones, portable multimedia devices or tablet computers. Alternatively, portable sensors with data recording and processing equipment may be used. Further, depending on the kind of activity test to be performed, the mobile device shall be adapted to display instructions for the subject regarding the activity to be carried out for the test. Particular envisaged activities to be carried out by the subject are described elsewhere herein and encompass the tests for active and passive gait and posture capabilities and cognitive capabilities as described in this specification.

Determining at least one performance parameter can be achieved either by deriving a desired measured value from the dataset as the performance parameter directly. Alternatively, the performance parameter may integrate one or more measured values from the dataset and, thus, may be a derived from the dataset by mathematical operations such as calculations. Typically, the performance parameter is derived from the dataset by an automated algorithm, e.g., by a computer program which automatically derives the performance parameter from the dataset of measurements when tangibly embedded on a data processing device feed by the said dataset.

The term “reference” as used herein refers to an identifier, which allows establishing a correlation between the determined at least on performance parameter and the EDSS. The reference is, typically, obtained from a computer-implemented regression model generated on training data, in an embodiment using random forest (RF) analysis, with the at least one performance parameters (Breiman 2001). The said training data are, typically, datasets of measurements of active and passive gait and posture capabilities and cognitive capabilities from subjects suffering from MS with known EDSS. The reference may be a model equation which allows to calculate the EDSS to be predicted form the determined at least one performance parameter. Alternatively, it may be a correlation curve or other graphical representation such as a scoring chart, at least one predictions plot, at least one correlations plot, and at least one residuals plot from which the EDSS to be predicted can be derived. A regression model may be established by analyzing the training data as referred above by RF using a processing unit in a data processing device such as a mobile device. The reference is, thus, typically a model equation, a scoring chart, at least one predictions plot, at least one correlations plot, and at least one residuals plot from the RF analysis.

Comparing the determined at least one performance parameter to a reference can be achieved by an automated comparison algorithm implemented on a data processing device such as a computer. The algorithm aims at deriving the predicted EDSS from the regression model. This can be done, e.g., by feeding the at least one performance parameter into a model equation or by comparing it to a correlation curve or other graphical representation. As a result of the comparison, the EDSS in the subject can be predicted.

The predicted EDSS is subsequently indicated to the subject or another person, such as a medical practitioner. Typically, this is achieved by displaying the predicted EDSS on a display of the mobile device or the evaluation device. Alternatively, a recommendation for a therapy, such as a drug treatment or for a certain life style, is provided automatically to the subject or other person. To this end, the predicted EDSS is compared to recommendations allocated to different EDSSs in a database. Once the predicted EDSS matches one of the stored and allocated EDSSs, a suitable recommendation can be identified due to the allocation of the recommendation to the stored diagnosis matching the predicted EDSS. Accordingly, it is, typically, envisaged that the recommendations and EDSSs are present in form of a relational database. However, other arrangements which allow for the identification of suitable recommendations are also possible and known to the skilled artisan.

Typically, the method of the present invention for predicting EDSS in a subject may be carried out as follows:

First, at least one performance parameter is determined from an existing dataset of measurements of active and passive gait and posture capabilities and cognitive capabilities obtained from said subject using a mobile device. Said dataset may have been transmitted from the mobile device to an evaluating device, such as a computer, or may be processed in the mobile device in order to derive the at least one performance parameter from the dataset.

Second, the determined at least one performance parameter is compared to a reference by, e.g., using a computer-implemented comparison algorithm carried out by the data processor of the mobile device or by the evaluating device, e.g., the computer. The said reference is obtained from a computer-implemented regression model generated on training data, in an embodiment using random forest (RF) analysis, with the at least one performance parameters. The result of the comparison is assessed with respect to the reference used in the comparison and based on the said assessment the EDSS of the subject will be automatically predicted.

Third, the EDSS is indicated to the subject or other person, such as a medical practitioner.

The invention, in light of the above, also specifically contemplates a method of predicting the EDSS in a subject suffering from MS comprising the steps of:
a) obtaining from said subject using a mobile device a dataset of measurements of active and passive gait and posture capabilities and cognitive capabilities during predetermined activity performed by the subject;
b) determining at least one performance parameter determined from a dataset of measurements obtained from said subject using a mobile device;
c) comparing the determined at least one performance parameter to a reference obtained from a computer-implemented regression model generated on training data, in an embodiment using random forest (RF) analysis, with the at least one performance parameters; and
d) predicting EDSS in said subject.

Advantageously, it has been found in the studies underlying the present invention that performance parameters obtained from datasets of measurements of active and passive gait and posture capabilities and cognitive capabilities in MS patients can be used as digital biomarkers for predicting the EDSS in those patients. The performance parameters can be compared to references obtained from a computer-implemented regression model generated on training data, in an embodiment using random forest (RF) analysis, with the at least one performance parameters. The said datasets can be acquired from the MS patients in a convenient manner by using mobile devices such as the omnipresent smart phones, portable multimedia devices or tablet computers on which the subjects perform certain tests rather than by complicated and subjective testing using the EDSS. The datasets acquired can be subsequently evaluated by the method of the invention for the performance parameter(s) suitable as digital biomarker. Said evaluation can be carried out on the same mobile device or it can be carried out on a separate remote device. Moreover, by using such mobile devices, recommendations on life style or therapy based on the predicted EDSS can be provided to the patients directly, i.e. without the consultation of a medical practitioner in a doctor's office or hospital ambulance. Thanks to the present invention, the life conditions of MS patients can be adjusted more precisely to the actual EDSS due to the use of actual determined performance parameters by the method of the invention. Thereby, therapeutic measures such as drug treatments or respiration support can be selected that are more efficient for the current status of the patient.

The method of the present invention may be used for:
assessing the disease condition;
monitoring patients, in particular, in a real life, daily situation and on large scale;
supporting patients with life style, support and/or therapy recommendations;
investigating drug efficacy, e.g. also during clinical trials;
facilitating and/or aiding therapeutic decision making;
supporting hospital managements;
supporting rehabilitation measure management;
improving the disease condition as a rehabilitation instrument stimulating higher density cognitive, motoric and walking activity
supporting health insurances assessments and management; and/or
supporting decisions in public health management.

The explanations and definitions for the terms made above apply mutatis mutandis to the embodiments described herein below.

In the following, particular embodiments of the method of the present invention are described:

In an embodiment, the said measurements of active and passive gait and posture capabilities and cognitive capabilities have been carried out using a mobile device.

In an embodiment, said mobile device is comprised in a smartphone, smartwatch, wearable sensor, portable multimedia device or tablet computer.

In yet another embodiment, said measurements of active and passive gait and posture capabilities and cognitive capabilities comprise measurements relating to movement characteristics, in particular, movement pattern or time required for performing a movement task, or accuracy, time or correctness of performing a cognitive task

In a further embodiment, at least 32 performance parameters are used.

In yet another embodiment, said reference obtained from a computer-implemented regression model generated on training data, in an embodiment using random forest (RF) analysis, with the at least one performance parameters is a model equation, a scoring chart, at least one predictions plot, at least one correlations plot, and at least one residuals plot from the analysis, in an embodiment RF analysis.

The present invention also contemplates a computer program, computer program product or computer readable storage medium having tangibly embedded said computer program, wherein the computer program comprises instructions when run on a data processing device or computer carry out the method of the present invention as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a dataset of pressure measurements obtained from the subject using a mobile, and
a data stream signal, typically encrypted, comprising the at least one performance parameter derived from the dataset of pressure measurements obtained from the subject using a mobile.

The present invention, further, relates to a method for determining at least one performance parameter from a dataset of measurements of active and passive gait and posture capabilities and cognitive capabilities from said subject suffering from MS using a mobile device
a) deriving at least one performance parameter from a dataset of measurements of active and passive gait and posture capabilities and cognitive capabilities from said subject using a mobile device; and
b) comparing the determined at least one performance parameter to a reference, said reference being obtained from a computer-implemented regression model generated on training data, in an embodiment using random forest (RF) analysis, with the at least one performance parameters,

wherein, typically, said at least one performance parameter can aid predicting the EDSS in said subject.

The present invention also encompasses a method for determining efficacy of a therapy against MS comprising the steps of the method of the invention (i.e. the method for predicting EDSS) and the further step of determining a therapy response if improvement of MS and/or EDSS occurs in the subject upon therapy or determining a failure of response if worsening of MS and/or EDSS occurs in the subject upon therapy or if MS and/or EDSS remains unchanged.

The term “a therapy against a MS” as used herein refers to all kinds of medical treatments, including drug-based therapies, respiratory support and the like. The term also encompasses, life-style recommendations and rehabilitation measures. Typically, the method encompasses recommendation of a drug-based therapy and, in particular, a therapy with a drug known to be useful for the treatment of MS. Such drug may be a therapy applying an anti-CD20 antibody and, more typically, Ocrelizumab (Hutas 2008). Moreover, the aforementioned method may comprise in yet another embodiment the additional step of applying the recommended therapy to the subject.

Moreover, encompassed in accordance with the present invention is a method for determining efficacy of a therapy against MS comprising the steps of the aforementioned method of the invention (i.e. the method for predicting EDSS) and the further step of determining a therapy response if improvement of MS and/or EDSS occurs in the subject upon therapy or determining a failure of response if worsening of MS and/or EDSS occurs in the subject upon therapy or if MS and/or EDSS remains unchanged.

The term “improvement” as referred to in accordance with the present invention relates to any improvement of the overall disease condition or of individual symptoms thereof and, in particular, the predicted EDSS. Likewise, a “worsening” means any worsening of the overall disease condition or individual symptoms thereof and, in particular, the predicted EDSS. Since, MS as a progressing disease is associated typically with a worsening of the overall disease condition and symptoms thereof, the worsening referred to in connection with the aforementioned method is an unexpected or untypical worsening which goes beyond the normal course of the disease. Unchanged MS means that the overall disease condition and the symptoms accompanying it are within the normal course of the disease.

Moreover, the present invention pertains to a method of monitoring MS in a subject comprising determining whether said disease improves, worsens or remains unchanged in a subject by carrying out the steps of the method of the invention (i.e. the method of predicting EDSS) at least two times during a predefined monitoring period. If the EDSS improves, the disease improves, if the EDSS worsens, the disease worsens and if the EDSS remains unchanged, the disease does as well.

The present invention relates to a mobile device comprising a processor, at least one sensor and a database as well as software which is tangibly embedded to said device and, when running on said device, carries out the method of the present invention.

The said mobile device is, thus, configured to be capable of acquiring the dataset and to determine the performance parameter therefrom. Moreover, it is configured to carry out the comparison to a reference and to establish the prediction, i.e. the prediction of the EDSS. Moreover, the mobile device may, typically, also be capable of obtaining and/or generating the reference from a computer-implemented regression model generated on training data, in an embodiment using random forest (RF) analysis, with the at least one performance parameters. Further details on how the mobile device can be designed for said purpose have been described elsewhere herein already in detail.

A system comprising a mobile device comprising at least one sensor and a remote device comprising a processor and a database as well as software which is tangibly embedded to said device and, when running on said device, carries out the method of the invention, wherein said mobile device and said remote device are operatively linked to each other.

Under “operatively linked to each other” it is to be understood that the devices are connected as to allow data transfer from one device to the other device. Typically, it is envisaged that at least the mobile device which acquires data from the subject is connected to the remote device carrying out the steps of the methods of the invention such that the acquired data can be transmitted for processing to the remote device. However, the remote device may also transmit data to the mobile device such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Further details may be found elsewhere in this specification. For data acquisition, the mobile device may comprise a user interface such as screen or other equipment for data acquisition. Typically, the activity measurements can be performed on a screen comprised by a mobile device, wherein it will be understood that the said screen may have different sizes including, e.g., a 5.1 inch screen.

Moreover, it will be understood that the present invention contemplates the use of the mobile device or the system according to the present invention for predicting the EDSS in a subject suffering from MS using at least one performance parameter from a dataset of measurements of active and passive gait and posture capabilities and cognitive capabilities from said subject.

The present invention also contemplates the use of the mobile device or the system according to the present invention for monitoring patients, in particular, in a real life, daily situation and on large scale.

Encompassed by the present invention is furthermore the use of the mobile device or the system according to the present invention for supporting patients with life style and/or therapy recommendations.

Yet, it will be understood that the present invention contemplates the use of the mobile device or the system according to the present invention for investigating drug safety and efficacy, e.g. also during clinical trials.

Further, the present invention contemplates the use of the mobile device or the system according to the present invention for facilitating and/or aiding therapeutic decision making.

Furthermore, the present invention provides for the use of the mobile device or the system according to the present invention for improving the disease condition as a rehabilitation instrument, and for supporting hospital management, rehabilitation measure management, health insurances assessments and management and/or supporting decisions in public health management.

In the following, further particular embodiments of the invention are listed:

Embodiment 1: A method for predicting the total motor score (EDSS) in a subject suffering from Multiple sclerosis (MS) comprising the steps of:
a) determining at least one performance parameter from a dataset of measurements of active and passive gait and posture capabilities and cognitive capabilities from said subject;
b) comparing the determined at least one performance parameter to a reference obtained from a computer-implemented regression model generated on training data, in an embodiment using random forest (RF) analysis, with the at least one performance parameters; and
c) predicting the EDSS of the subject based on said comparison.

Embodiment 2: The method of embodiment 1, wherein the said measurements of active and passive gait and posture capabilities and cognitive capabilities have been carried out using a mobile device, in an embodiment wherein the measurements of active and passive gait and posture capabilities and cognitive capabilities are carried out using a mobile device.

Embodiment 3: The method of embodiment 2, wherein said mobile device is comprised in a smartphone, smartwatch, wearable sensor, portable multimedia device or tablet computer.

Embodiment 4: The method of any one of embodiments 1 to 3, wherein said measurements of active and passive gait and posture capabilities and cognitive capabilities comprise measurements relating to movement characteristics, in particular, movement pattern or time required for performing a movement task, or accuracy, time or correctness of performing a cognitive task.

Embodiment 5: The method of any one of embodiments 1 to 4, wherein at least 32 performance parameters are used.

Embodiment 6: The method of any one of embodiments 1 to 5, wherein at least three, in an embodiment at least four, in a further embodiment at least six, performance parameters of Table 1 are determined, in an embodiment wherein at least the first three, in an embodiment the first four, in a further embodiment the first six, performance parameters of Table 1 are determined.

Embodiment 7. The method of any one of embodiments 1 to 2, wherein all performance parameters of Table 1 are determined.

Embodiment 8. The method of any one of embodiments 1 to 7, wherein the at least one performance parameter of step a) is derived from the dataset by an automated algorithm tangibly embedded on a data processing device.

Embodiment 9. The method of any one of embodiments 1 to 8, wherein comparing the at least one performance parameter to a reference in step b) is achieved by an automated comparison algorithm implemented on a data processing device.

Embodiment 10. The method of any one of embodiments 1 to 9, wherein said reference obtained from a computer-implemented regression model generated on training data, in an embodiment using random forest (RF) analysis, with the at least one performance parameters is a model equation, a scoring chart, at least one predictions plot, at least one correlations plot, and at least one residuals plot from the analysis, in an embodiment the RF analysis.

Embodiment 11. The method of any one of embodiments 1 to 10, wherein said method is computer-implemented.

Embodiment 12: A mobile device comprising a processor, at least one sensor and a database as well as software which is tangibly embedded to said device and, when running on said device, carries out at least step a) of the method of any one of embodiments 1 to 11, in an embodiment carries out the method of any one of embodiments 1 to 11.

Embodiment 13: A system comprising a mobile device comprising at least one sensor and a remote device comprising a processor and a database as well as software which is tangibly embedded to said device and, when running on said device, carries out the method of any one of embodiments 1 to 11, wherein said mobile device and said remote device are operatively linked to each other.

Embodiment 14: Use of the mobile device according to embodiment 12 or the system of embodiment 13 for predicting the EDSS in a subject suffering from MS using at least one performance parameter from a dataset of measurements of active and passive gait and posture capabilities and cognitive capabilities from said subject.

All references cited throughout this specification are herewith incorporated by reference with respect to their entire disclosure content and with respect to the specific disclosure contents mentioned in the specification.

### BRIEF DESCRIPTION OF THE FIGURES

FIG. 1 shows EDSS prediction results obtained with different models, i.e. k nearest neighbors (kNN); linear regression; partial last-squares (PLS); random forest (RF); and extremely randomized Trees (XT); f: number of features included in model, y-axis: rₛ (correlation between predicted and actual values); upper row: test data set, lower row: training data; in the lower row, upper graphs relate to “mean” prediction, i.e. the prediction on the average value of all observations per subject, and the lower graphs relate to “all” prediction, i.e. prediction on all individual observations; the best result is obtained using RF.

### EXAMPLES

The following Examples merely illustrate the invention. Whatsoever, they shall not be construed in a way as to limit the scope of the invention.

### Example 1: Results from the Prospective Pilot Study (FLOODLIGHT) to Evaluate the Feasibility of Conducting Remote Patient Monitoring with the Use of Digital Technology in Patients with Multiple Sclerosis

A study population was selected by using the following inclusion and exclusion criteria:

Key inclusion criteria:

Signed informed consent form

Able to comply with the study protocol, in the investigator's judgment

Age 18-55 years, inclusive

Have a definite diagnosis of MS, confirmed as per the revised McDonald 2010 criteria

EDSS score of 0.0 to 5.5, inclusive

Weight: 45-110 kg

For women of childbearing potential: Agreement to use an acceptable birth control method during the study period

Key exclusion criteria:

Severely ill and unstable patients as per investigator's discretion

Change in dosing regimen or switch of disease modifying therapy (DMT) in the last 12 weeks prior to enrollment

Pregnant or lactating, or intending to become pregnant during the study

It is a primary objective of this study to show adherence to smartphone and smartwatch-based assessments quantified as compliance level (%) and to obtain feedback from patients and healthy controls on the smartphone and smartwatch schedule of assessments and the impact on their daily activities using a satisfaction questionnaire. Furthermore, additional objectives are addressed, in particular, the association between assessments conducted using the Floodlight Test and conventional MS clinical outcomes was determined, it was established if Floodlight measures can be used as a marker for disease activity/progression and are associated with changes in MRI and clinical outcomes over time and it was determined if the Floodlight Test Battery can differentiate between patients with and without MS, and between phenotypes in patients with MS.

In addition to the active tests and passive monitoring, the following assessments were performed at each scheduled clinic visit:
Oral Version of SDMT
Fatigue Scale for Motor and Cognitive Functions (FSMC)
Timed 25-Foot Walk Test (T25-FW)
Berg Balance Scale (BBS)
9-Hole Peg Test (9HPT)
Patient Health Questionnaire (PHQ-9)
Patients with MS only:
Brain MRI (MSmetrix)
Expanded Disability Status Scale (EDSS)
Patient Determined Disease Steps (PDDS)
Pen and paper version of MSIS-29

While performing in-clinic tests, patients and healthy controls were asked to carry/wear smartphone and smartwatch to collect sensor data along with in-clinic measures.

In summary, the results of the study showed that patients are highly engaged with the smartphone- and smartwatch-based assessments. Moreover, there is a correlation between tests and in-clinic clinical outcome measures recorded at baseline which suggests that the smartphone-based Floodlight Test Battery shall become a powerful tool to continuously monitor MS in a real-world scenario. Further, the smartphone-based measurement of turning speed while walking and performing U-turns appeared to correlate with EDSS.

### Example 2: Analysis of the Floodlight Study Using a Machine Learning Algorithm

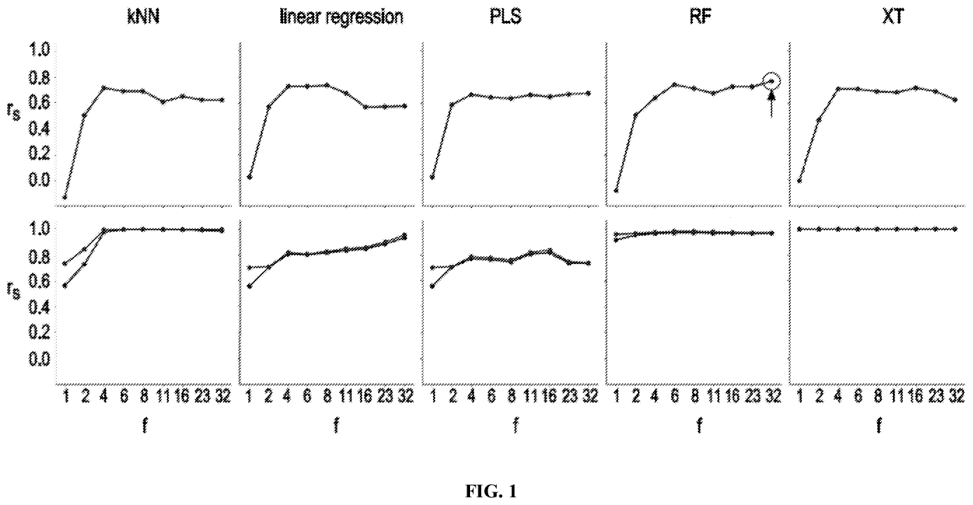

Data from Floodlight POC study from 52 subjects were investigated by kNN, linear regression, PLS, RF and XT. In total, 889 features from 7 tests were evaluated during model building. The tests used for this prediction were the Symbol-Digits Modalities Test (SMDT) where the subject has to match as many symbols as possible to digits in a given time span; the pinching test, where the subject has to squeeze, using the thumb and index finger, as many tomatoes shown on the screen as possible in a given time span; the Draw-A-Shape test, where the subject has to trace shapes on the screen; the Standing Balance Test where the subject has to stand upright for 30 seconds; the 5 U-Turn test where the subject has to walk short spans followed by 180 degree turns; the 2 Minute Walking test, where the subject has to walk for two minutes; and finally the passive monitoring of the gait. The models build by the different techniques were investigated by a machine learning algorithm in order to identify the model with the best correlation. FIG. 1 show a correlations plot for analysis models, in particular regression models, for predicting an expanded disability status scale value indicative of multiple sclerosis. FIG. 1, in particular, shows the Spearman correlation coefficient rₛ between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. It was found that the best performing regression model is RF with 32 features included in the model, having an rₛ value of 0.77, indicated with circle and arrow. The following table gives an overview for features from the RF algorithm (best correlation), test from which the feature was derived, short description of feature and ranking:

| feature | test | Description of feature | rank |
|---|---|---|---|
| logistic step_power_mean | Passive | Average per-step power coefficient | 1 |
| (40-60 s) | Monitoring | (integral of variance in accelerometer | |
| | | radius over per-step time span) for | |
| | | gait bouts spanning 40-60 s | |
| sigmoid turns_utt | U-TURN | Number of turns | 2 |
| log10 Gc_0_15 | SDMT | Mean Timegap between correct | 3 |
| | | responses from time 0 to 15 seconds | |
| sigmoid | U-TURN | maximum turn speed | 4 |
| turn_speed_max_utt | | | |
| logistic step_power_mean | 2MWT | Average per-step power coefficient | 5 |
| | | (integral of variance in accelerometer | |
| | | radius over per-step time span) | |
| sigmoid turn_speed_min_utt | U-TURN | minimum turn speed | 6 |
| sigmoid | Passive | Variance of per-step power coefficient | 7 |
| step_power_variance | Monitoring | for gait bouts spanning 60-90 s | |
| (60-90 s) | | | |
| logistic | Passive | Variance of per-step power coefficient | 8 |
| step_power_variance | Monitoring | for gait bouts spanning 40-60 s | |
| (40-60 s) | | | |
| sigmoid step_power_mean | Passive | Average per-step power coefficient | 9 |
| (<20 s) | Monitoring | (integral of variance in accelerometer | |
| | | radius over per-step time span) for | |
| | | gait bouts spanning <20 s | |
| span_duration_s_median_utt | U-TURN | median gait bout length | 10 |
| logistic | Passive | Variance of per-step power coefficient | 11 |
| step_power_variance | Monitoring | for gait bouts spanning 20-40 s | |
| (20-40 s) | | | |
| sigmoid | Passive | Variance of per-step power coefficient | 12 |
| step_power_variance | Monitoring | for gait bouts spanning 90-120 s | |
| (90-120 s) | | | |
| sigmoid | U-TURN | median turn speed | 13 |
| turn_speed_median_utt | | | |
| logistic step_power_mean | Passive | Average per-step power coefficient | 14 |
| (60-90 s) | Monitoring | (integral of variance in accelerometer | |
| | | radius over per-step time span) for | |
| | | gait bouts spanning 60-90 s | |
| sigmoid GcM_0_15 | SDMT | Maximal Timegap between correct | 15 |
| | | responses from time 0 to 15 seconds | |
| logistic step_power_mean | Passive | Average per-step power coefficient | 16 |
| (20-40 s) | Monitoring | (integral of variance in accelerometer | |
| | | radius over per-step time span) for | |
| | | gait bouts spanning 20-40 s | |
| logistic step_power_mean | Passive | Average per-step power coefficient | 17 |
| (90-120 s) | Monitoring | (integral of variance in accelerometer | |
| | | radius over per-step time span) for | |
| | | gait bouts spanning 90-120 s | |
| CCR_0_45 | SDMT | from time 0 to 45 seconds: Number of | 18 |
| | | correct responses within the longest | |
| | | sequence of overall consecutive | |
| | | correct responses | |
| span_duration_s_max_utt | U-TURN | maximum gait bout length | 19 |
| log10 R_Symbol_9 | SDMT | Number of total responses for symbol | 20 |
| | | 9: “.-” | |
| Gc_0_30 | SDMT | Mean Timegap between correct | 21 |
| | | responses from time 0 to 30 seconds | |
| sigmoid CCR_0_15 | SDMT | from time 0 to 15 seconds: Number of | 22 |
| | | correct responses within the longest | |
| | | sequence of overall consecutive | |
| | | correct responses | |
| sigmoid GM_0_15 | SDMT | Maximal Timegap between responses | 23 |
| | | from time 0 to 15 seconds | |
| sigmoid R_0_15 | SDMT | Number of total responses from time 0 | 24 |
| | | to 15 seconds | |
| log10 CR_Symbol_8 | SDMT | Number of correct responses for | 25 |
| | | symbol 8: “)” | |
| log10 CCR_0_30 | SDMT | from time 0 to 30 seconds: Number of | 26 |
| | | correct responses within the longest | |
| | | sequence of overall consecutive | |
| | | correct responses | |
| log10 G_0_15 | SDMT | Mean Timegap between responses | 27 |
| | | from time 0 to 15 seconds | |
| sigmoid CR_0_15 | SDMT | Number of correct responses from | 28 |
| | | time 0 to 15 seconds | |
| log10 Gc_0_45 | SDMT | Mean Timegap between correct | 29 |
| | | responses from time 0 to 45 seconds | |
| log10 R_Symbol_8 | SDMT | Number of total responses for symbol | 30 |
| | | 8: “)” | |
| log10 R_0_30 | SDMT | Number of total responses from time 0 | 31 |
| | | to 30 seconds | |
| sigmoid CR_0_30 | SDMT | Number of correct responses from | 32 |
| | | time 0 to 30 seconds | |

These features will be used to identify EDSS values using data from test subjects and the RF analysis.

### CITED REFERENCES

Aktas 2005, Neuron 46, 421-432.
Zamvil 2003, Neuron 38:685-688.
Kurtzke 1983, Neurology. 33 (11): 1444-52. doi:10.1212/WNL.33.11.1444. PMID 6685237.
Collins 2016, Multiple Sclerosis. 22 (10): 1349-58. doi:10.1177/1352458515616205. PMC 5015760. PMID 26564998.
Breiman 2001, Machine Learning 45(1): 5-32 doi:10.1023/A:1010933404324
Bove 2015, Neurol Neuroimmunol Neuroinflamm 2 (6):e162).
Link 2006, J Neuroimmunol. 180 (1-2): 17-28.
Tsang 2011, Australian family physician 40 (12): 948-55.
Compston 2008, Lancet 372(9648): 1502-17.
Johnston 2012, Drugs 72 (9): 1195-211.
Hutas 2008, Current opinion in investigational drugs 9 (11):1206-15.

1. A method for predicting the total motor score (EDSS) in a subject suffering from Multiple sclerosis (MS) comprising the steps of:
a) determining at least one performance parameter from a dataset of measurements of active and passive gait and posture capabilities and cognitive capabilities from said subject;
b) comparing the determined at least one performance parameter to a reference obtained from a computer-implemented regression model generated on training data using random forest (RF) analysis with the at least one performance parameters; and
c) predicting the EDSS of the subject based on said comparison.

2. The method of claim 1, wherein the said measurements of active and passive gait and posture capabilities and cognitive capabilities have been carried out using a mobile device, in an embodiment wherein the measurements of active and passive gait and posture capabilities and cognitive capabilities are carried out using a mobile device.

3. The method of claim 2, wherein said mobile device is comprised in a smartphone, smartwatch, wearable sensor, portable multimedia device or tablet computer.

4. The method of claim 1, wherein said measurements of active and passive gait and posture capabilities and cognitive capabilities comprise measurements relating to movement characteristics, in particular, movement pattern or time required for performing a movement task, or accuracy, time or correctness of performing a cognitive task.

5. The method of claim 1, wherein at least 32 performance parameters are used.

6. The method of claim 1, wherein at least three performance parameters of Table 1 are determined.

7. The method of claim 1, wherein all performance parameters of Table 1 are determined.

8. The method of claim 1, wherein the at least one performance parameter of step a) is derived from the dataset by an automated algorithm tangibly embedded on a data processing device.

9. The method of claim 1, wherein comparing the at least one performance parameter to a reference in step b) is achieved by an automated comparison algorithm implemented on a data processing device.

10. The method of claim 1, wherein said reference obtained from a computer-implemented regression model generated on training data using random forest (RF) analysis with the at least one performance parameters is a model equation, a scoring chart, at least one predictions plot, at least one correlations plot, and at least one residuals plot from the RF analysis.

11. The method of claim 1, wherein said method is computer-implemented.

12. A mobile device comprising a processor, at least one sensor and a database as well as software which is tangibly embedded to said device and, when running on said device, carries out at least step a) of the method of claim 1.

13. A system comprising a mobile device comprising at least one sensor and a remote device comprising a processor and a database as well as software which is tangibly embedded to said device and, when running on said device, carries out the method of claim 1, wherein said mobile device and said remote device are operatively linked to each other.

14. Use of the mobile device according to claim 12 for predicting EDSS in a subject suffering from MS using at least one performance parameter from a dataset of measurements of active and passive gait and posture capabilities and cognitive capabilities from said subject.
